(19) United States
(12) Patent Application Publication
(10) Pub. No.: US 2006/0142807 A1
(43) Pub. Date: Jun. 29, 2006

(54) Method and article for packaging an automatic external defibrillator for use without a prescription

(76) Inventors: Wendy Katzman, Seattle, WA (US); Jesse Warwick, Shoreline, WA (US); Teresa Skarr, Snohomish, WA (US); Tamara Yount, Snohomish, WA (US); Brian Packard, North Andover, MA (US); Daniel J. Powers, Issaquah, WA (US); David E. Snyder, Bainbridge Island, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(21) Appl. No.: 11/109,928

(22) Filed: Apr. 19, 2005

Related U.S. Application Data
(60) Provisional application No. 60/639,476, filed on Dec. 27, 2004.

(51) Int. Cl. A61N 1/39

(52) U.S. Cl. 607/5

(57) ABSTRACT

A method and article for packaging an automatic external defibrillator for use without a prescription are described. The OTC AED is encased in a rigid polymeric disclosure which is a distinctive color or set of colors to enable the OTC AED to be readily distinguished in the home environment. The defibrillator is packaged in a box with multicolored depictions of the defibrillator on multiple sides of the box, which enable the distinctive coloring of the defibrillator to be readily identified by the exterior of the packaging. The packaging may also include indication of use for the OTC AED and considerations useful to a layperson purchaser when deciding whether to acquire an OTC AED.

This application claims the benefit of Provisional U.S. patent application Ser. No. 60/639,476, filed Dec. 27, 2004.

This invention relates to automatic external defibrillators (AEDs) and, in particular, to AEDs which can be sold to individuals over the counter (OTC) without a prescription.

Automatic external defibrillators have been in use for a number of years to treat individuals stricken with sudden cardiac arrest, one of the largest causes of death in the United States. Sudden cardiac arrest (SCA) most often occurs without warning, striking people with no previously recognized symptoms of heart disease. It is estimated that more than 1000 people per day are victims of sudden cardiac arrest in the United States alone. SCA results when the electrical component of the heart no longer functions properly causing an abnormal sinus rhythm. One such abnormal sinus rhythm, ventricular fibrillation (VF), is caused by abnormal and very fast electrical activity in the heart. As a result, the heart fails to adequately pump blood through the body. VF may be treated by applying an electric shock to a patient's heart through the use of a defibrillator. The shock clears the heart of abnormal electrical activity (in a process called “defibrillation”) by producing a momentary asystole and providing an opportunity for the heart's natural pacemaker areas to restore normal rhythmic function. When delivered external to the patient, these electrical pulses are high energy pulses, typically in the range of 30 to 360 Joules of energy.

Defibrillators have undergone an evolution over the past decade. Originally defibrillators were manual devices requiring both medical and technical expertise to operate. A physician would carefully set the controls of the defibrillator to apply a shock which diagnosis of the patient or experience with other patients in similar conditions indicated to be most likely to be effective. Following many years of experience with manual defibrillators and motivated by advances in microprocessing and signal analysis, defibrillators have become more automated to the point where a two-pad electrode attached to a patient's chest can detect and diagnose VF and deliver an appropriate shock through the chest wall. However such automated defibrillators continued to be prescription devices used by medical professionals or under the auspices of a controlled emergency response program as described in U.S. Pat. No. 6,694,299. In the final months of 2004 AEDs have reached a level of sophistication and reliability which now enables them to be sold to laypersons without prescription, as over-the-counter (OTC) medical devices. AEDs may now be sold through retail channels (stores, websites, catalogs) and purchased by anyone for use at home in the event of a sudden cardiac arrest emergency.

The use of OTC AEDs poses new demands on the packaging of such AEDs, since the AEDs are no longer being purchased through a doctor's prescription and oversight. Since OTC AEDs are medical devices with the ability to deliver an electrical shock of several thousand volts (in dosages generally measured in joules) it is important that the packaging for the AED be clearly marked. In the past, AEDs were commonly shipped to hospital and emergency response medical professionals in plain cardboard boxes with alphanumeric identification, with the knowledge that this was sufficient for the professional user. OTC AEDs require different identification that quickly conveys necessary information visually to laypersons.

In accordance with the principles of the present invention a method and article for packaging an AED for use without a prescription are described. In one embodiment the packaging includes multicolor graphical images of the distinctively colored OTC AED prominently displayed on multiple sides of the packaging. In another embodiment the packaging conveys information which is useful to the layperson purchaser in deciding whether to purchase the OTC AED. In another embodiment the packaging alerts the purchaser on the exterior of the packaging to the importance of setting up the AED for a readiness state prior to emergency use. In another embodiment the AED and/or its carrying case prominently display a number by which the layperson rescuer can contact professional medical assistance, such as the 911 emergency telephone number. These and other attributes of embodiments of the present invention will become apparent from the following detailed description and drawings in which:

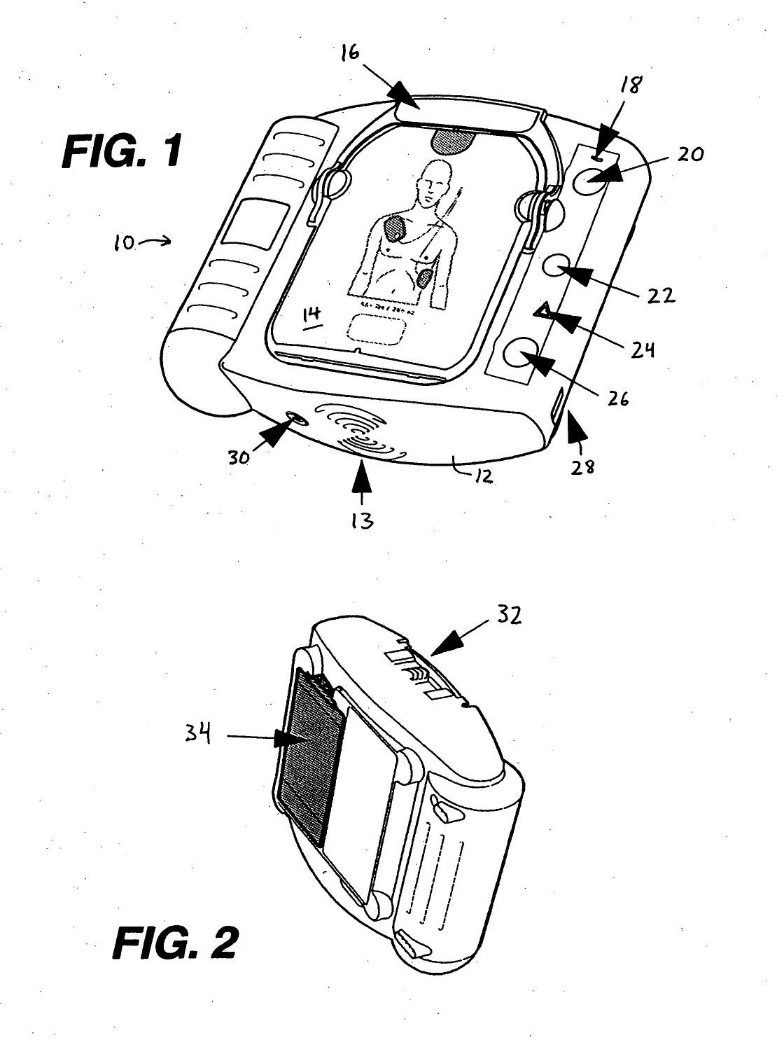

FIG. 1 illustrates a top perspective view of an OTC automatic external defibrillator.

FIG. 2 illustrates a bottom perspective view of the OTC automatic external defibrillator of FIG. 1.

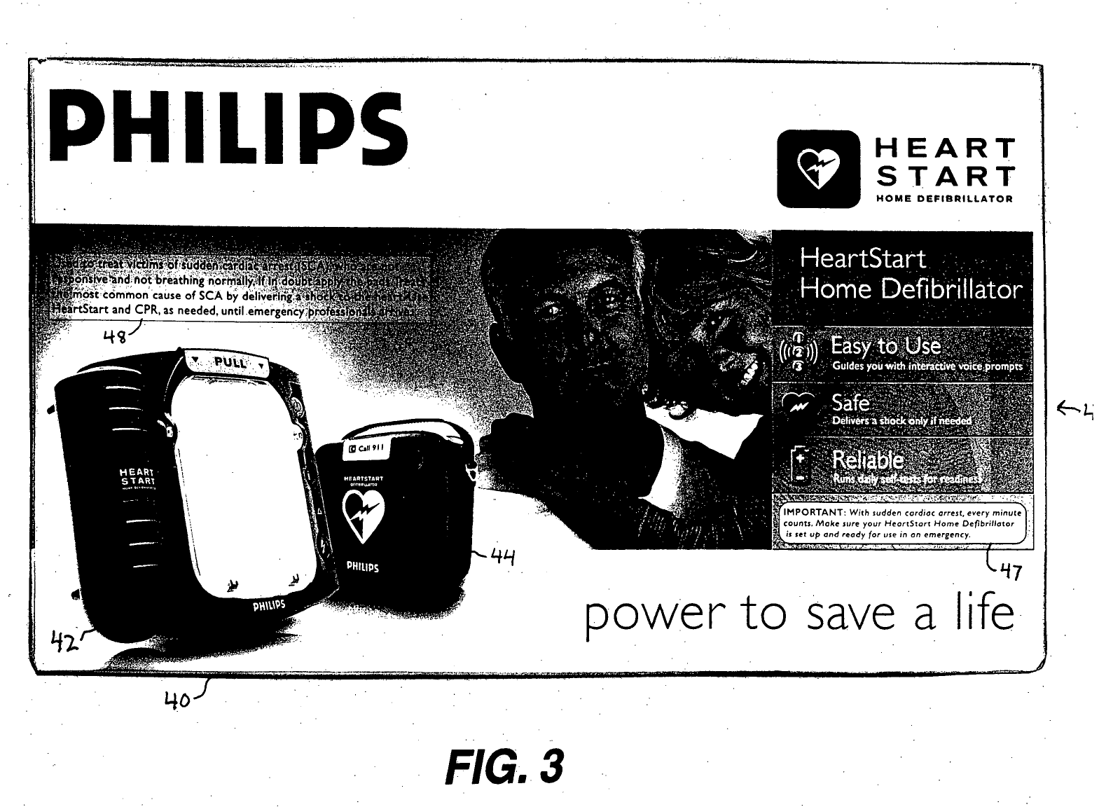

FIG. 3 is a top plan view of packaging for an OTC automatic external defibrillator in accordance with an embodiment of the present invention.

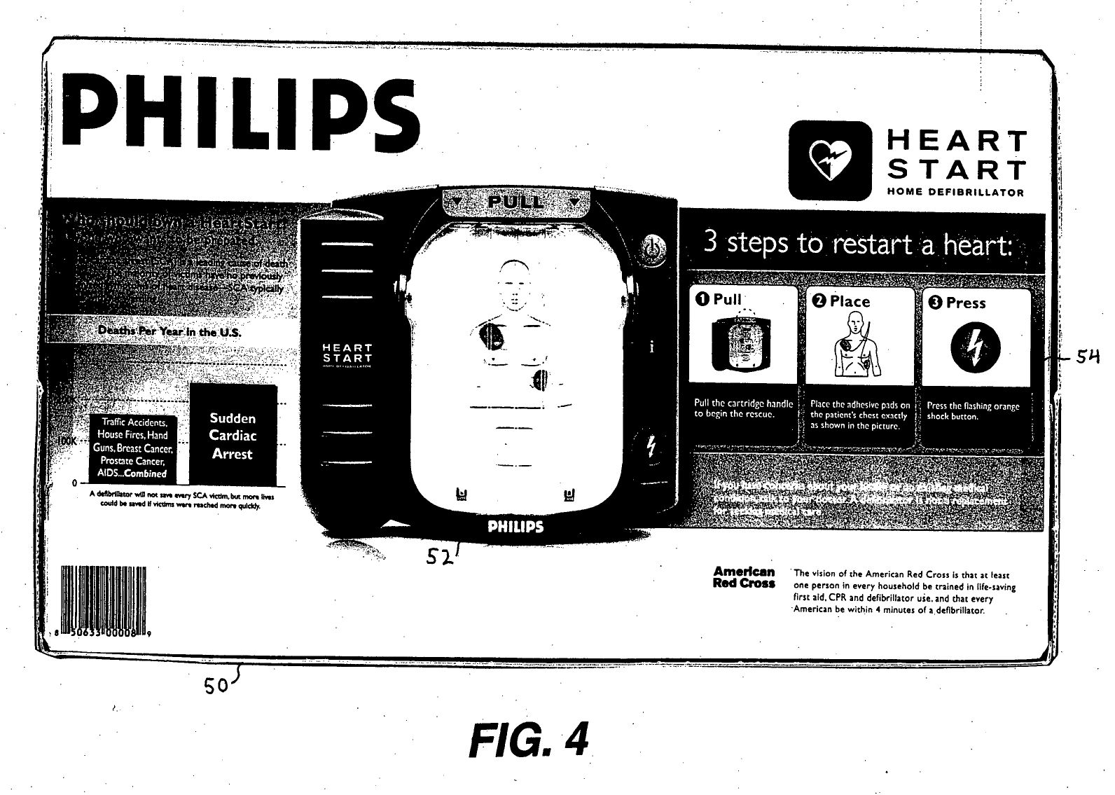

FIG. 4 is a bottom plan view of packaging for an OTC automatic external defibrillator in accordance with an embodiment of the present invention.

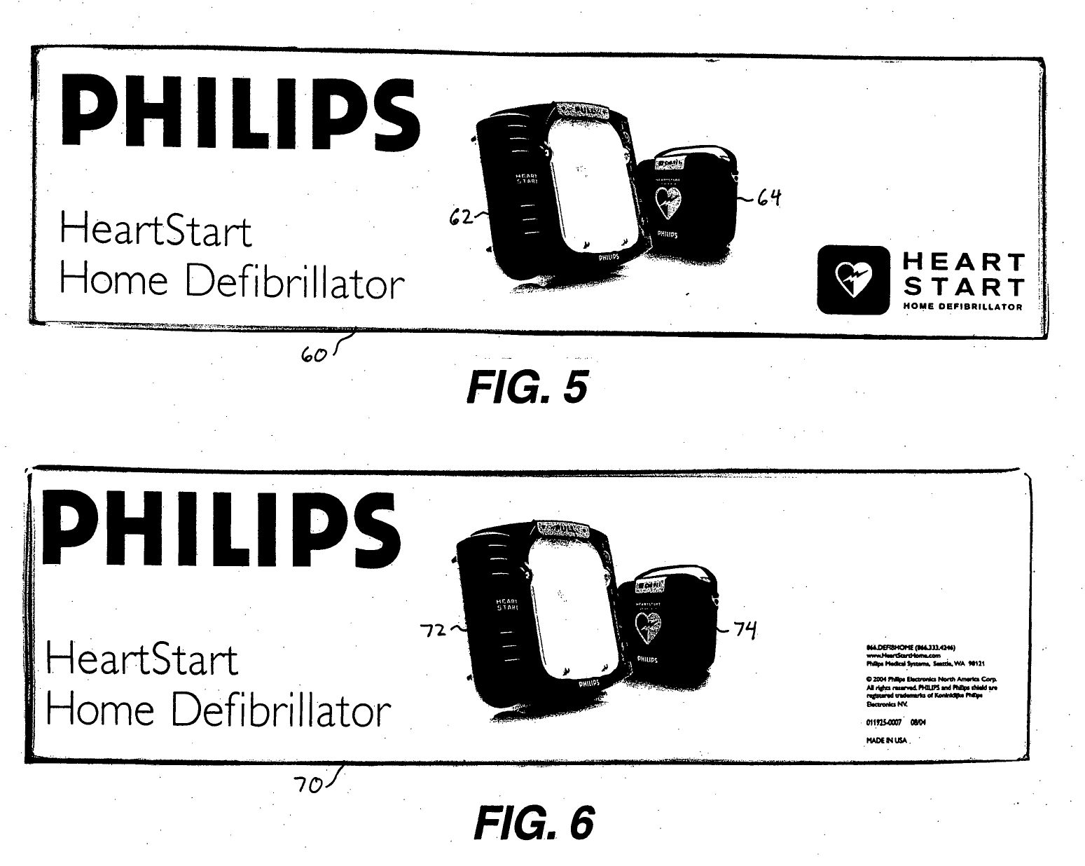

FIGS. 5 and 6 are side plan views of packaging for an OTC automatic external defibrillator in accordance with an embodiment of the present invention.

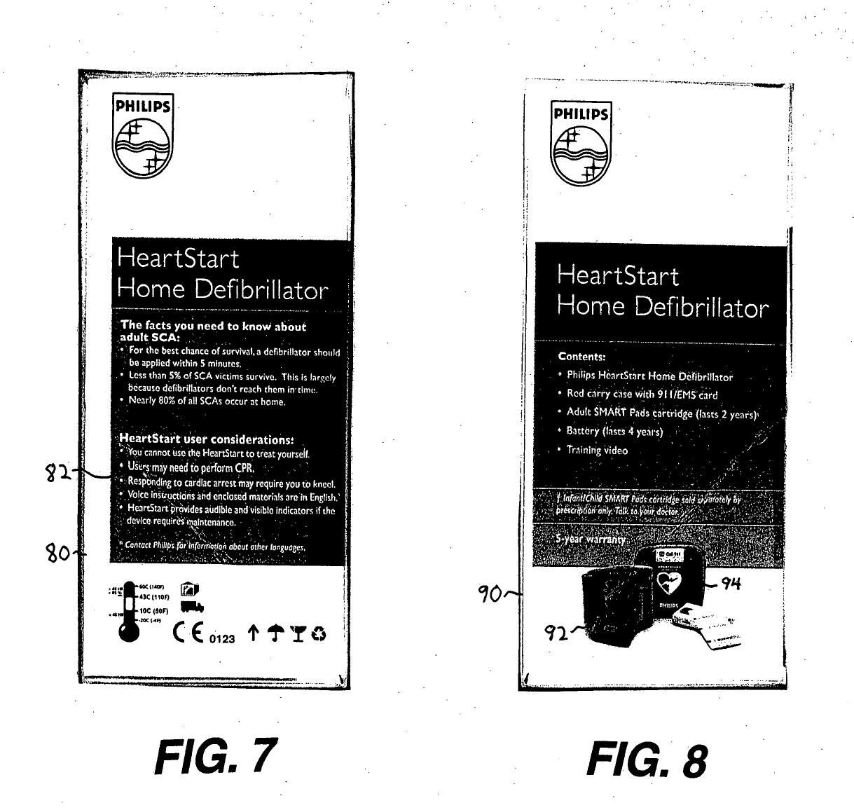

FIGS. 7 and 8 are end plan views of packaging for an OTC automatic external defibrillator in accordance with an embodiment of the present invention.

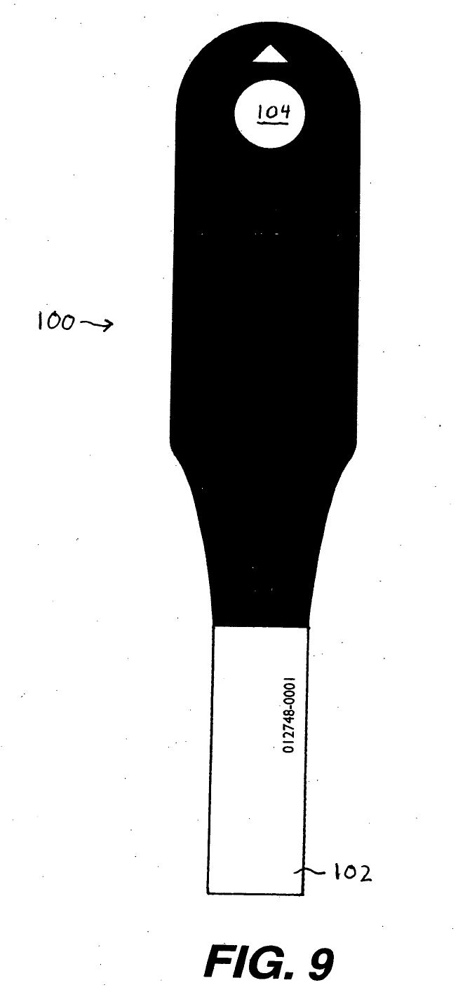

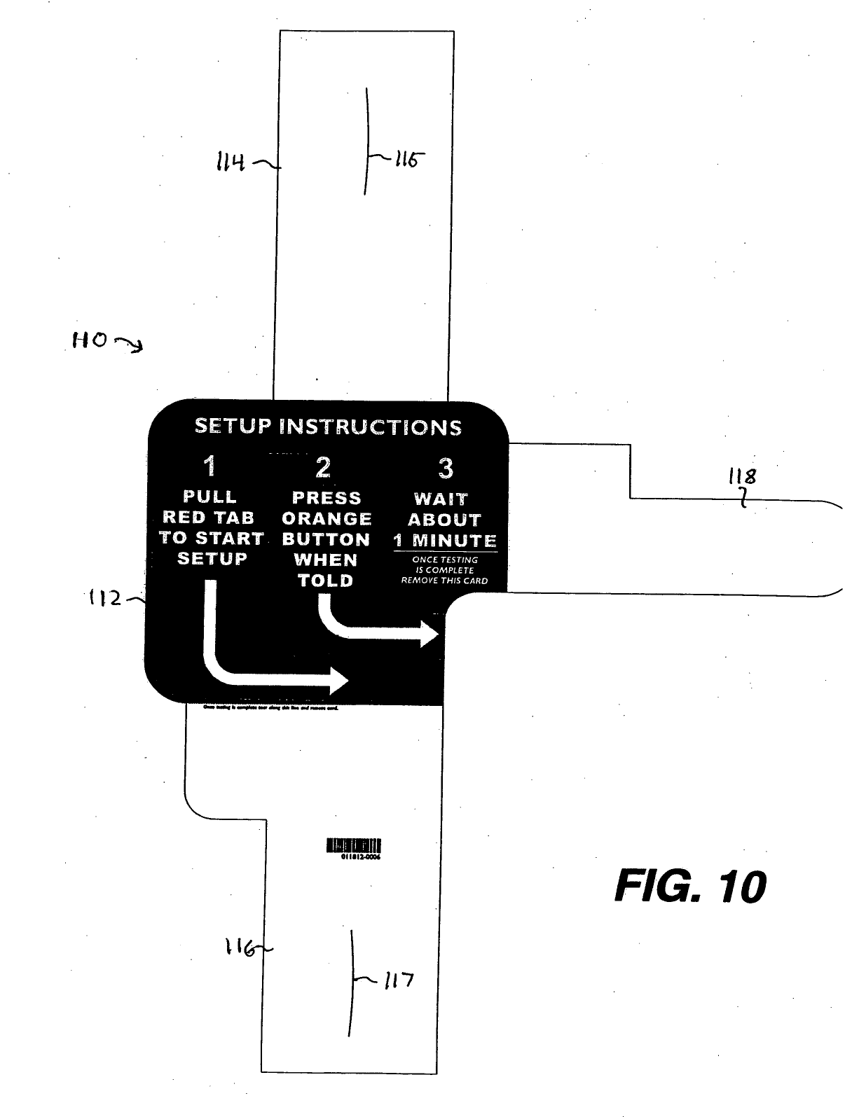

FIG. 9 illustrates a pull tab which connects the battery to an OTC AED and initiates the setup procedure.

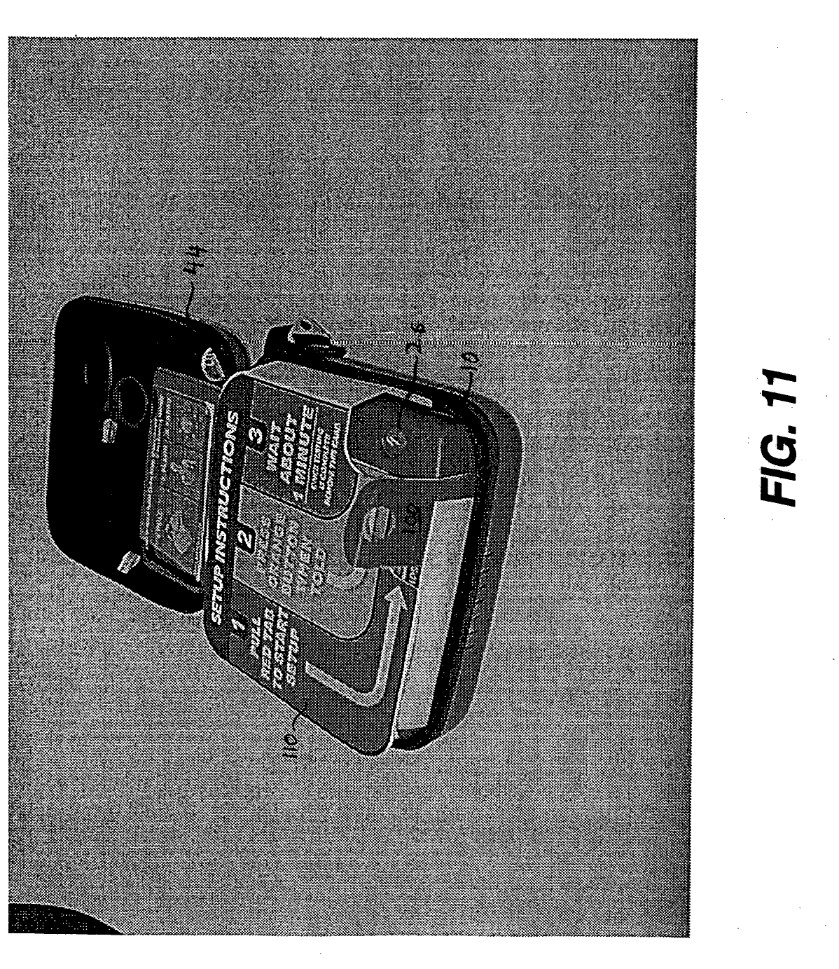

FIGS. 10 and 11 illustrate a shipping cover which facilitates the setup procedure.

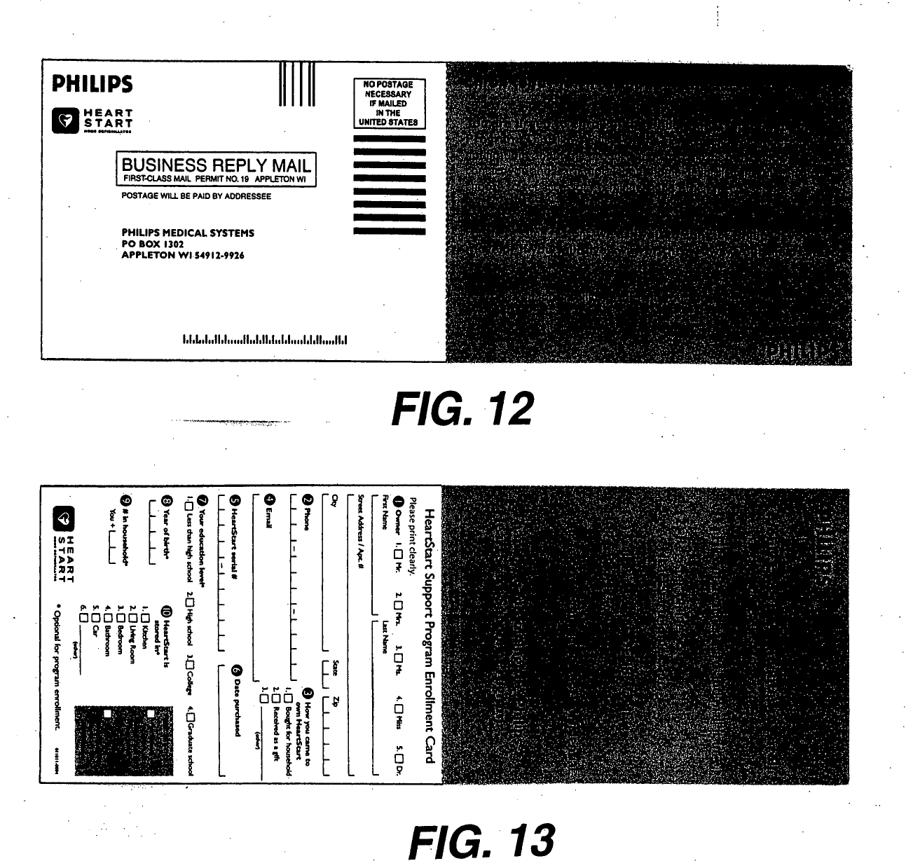

FIGS. 12 and 13 illustrate the front and back of an OTC AED support program enrollment card.

Referring first to FIG. 1, an OTC AED 10 is shown in a top perspective view. The OTC AED 10 is housed in a rugged polymeric case 12 which protects the electronic circuitry inside the case and also protects the layperson user from shocks. In this embodiment the case is colored a distinctive color which readily identifies the OTC AED to the layperson user, such as red, yellow, orange, green, blue, black, or combinations thereof. Other suitable distinctive colors are light green, silver gray, and various shades of white or off-white. Combinations of the aforementioned colors also provide distinctive colorings to the layperson user such as yellow/black, yellow/blue, yellow/white gold/black, blue/silver, yellow/blue/black, and blue/white/black. Other distinctive colors include the yellows, oranges and reds often used for traffic signs and signals. It is important in the home environment that the OTC AED be marked by a prominent color or colors so as to be immediately recognized by a potential rescuer in the event of a home cardiac emergency. Unlike airports and public facilities where AEDs are generally mounted in distinctive locations such as on walls in high traffic areas and with signage to mark and indicate their locations, an OTC AED may be placed anywhere in the home. Since a home OTC AED may go an extended period of time without use, it may be stored in a location lacking prominence such as in a closet or drawer. Accordingly it is very important for the OTC AED to bear a distinctive color as this may be the primary means by which a rescuer can quickly locate the OTC AED in the home during an emergency. The OTC AED may be stored when not in use in a carrying case which may be a distinctive color such as red, black, navy blue, or blue/yellow color.

Attached to the case 12 by electrical leads are a pair of electrode pads. In the embodiment of FIG. 1 the electrode pads are in a sealed airtight cartridge 14 located in a recess on the top side of the OTC AED 10. The electrode pads are accessed for use by pulling up on a handle 16 which allows removal of a plastic cover over the electrode pads. A small ready light 18 informs the user of the readiness of the OTC AED. In this embodiment the ready light blinks after the OTC AED has been properly set up and is ready for use. The ready light is on constantly when the OTC AED is in use, and the ready light is off when the OTC AED needs attention.

Below the ready light is an on/off button 20. The on/off button is pressed to turn on the OTC AED for use. To turn off the OTC AED a user holds the on/off button down for one second or more. An information button 22 flashes when information is available for the user. The user depresses the information button to access the available information. A caution light 24 blinks when the OTC AED is acquiring heartbeat information from the patient and lights continuously when a shock is advised, alerting the rescuer and others that no one should be touching the patient during these times. Interaction with the patient while the heart signal is being acquired can introduce unwanted artifacts into the detected ECG signal. A shock button 26 is depressed to deliver a shock after the OTC AED informs the rescuer that a shock is advised. An infrared port 28 on the side of the OTC AED is used to transfer data between the OTC AED and a computer. This data port find used after a patient has been rescued and a physician desires to have the OTC AED event data downloaded to his or her computer for detailed analysis.

A speaker 13 provides voice instructions to a rescuer to guide the rescuer through the use of the OTC AED to treat a patient. A beeper 30 is provided which “chirps” when the OTC AED needs attention such as electrode pad replacement or a new battery.

FIG. 2 illustrates another view of the OTC AED 10 in which a cartridge latch 32 is seen on the upper end of the OTC AED. When this latch is pushed to the right the electrode pad cartridge is released from its recess in the OTC AED case 12. The cartridge latch 32 is used when an electrode pad cartridge is to be replaced or exchanged for a training pad set for training on the OTC AED. On the back of the OTC AED case is a battery compartment which houses a battery 34 that powers the OTC AED. In this embodiment the battery 34 is a disposable battery. When the battery 34 becomes discharged, generally after about four years in the readiness state, it is replaced with a fresh battery.

In this embodiment the OTC AED contains extensive self-test circuitry which automatically monitors the state of various parts of the OTC AED on a regular basis. Self-test circuitry is very important for an OTC AED because it cannot be expected that purchasers of the OTC AED will adhere to any formal maintenance schedule for the OTC AED. One component that is self-tested in this embodiment is the electrode pad set. The electrode pads include an adhesive gel which adheres the electrodes to the patient and provides good electrical conductivity with the patient. This adhesive gel is hydrophilic and over time can become subject to desiccation which reduces the effectiveness of the pads. In the hospital setting or the medical emergency responder setting electrode pads are generally used in a relatively short time-frame and desiccation is often not a problem. In addition, these medical professionals are generally more cognizant of the need for attention to expiration dates and other maintenance to their medical equipment. Electrode pads for the prescription defibrillators used by these medical professionals are often not connected to the defibrillator until the defibrillator is to be used and thus cannot be tested by the AED prior to use. Organizations such as airports and office buildings which have deployed defibrillators generally do so under the direction of a medical officer who oversees a maintenance program for the defibrillators. In the home environment it is to be expected that an OTC AED may sit in readiness for the full two-year anticipated lifetime of a typical electrode pad set without being inspected or used. Accordingly, in one embodiment of the present invention the electrode pads are normally electrically connected to self-test circuitry while the OTC AED is in the readiness state. With an electrode pad cartridge this can be done by embedding conductors in the wall of the cartridge. The electrode pad leads inside the cartridge are connected to these conductors, which enables electrical connectivity to the exterior of an air-tight sealed cartridge. The cartridge conductors engage mating conductors in the recess of the OTC AED case, thereby putting the electrode pads into electrical communication with the OTC AED self-test circuitry. This permits the electrode pads to be automatically tested by the OTC AED on a periodic basis. If the self-testing determines that the electrode pads have dried out or suffered some other detected deterioration the user is alerted to replace the pads by the chirping of the beeper 30 and the absence of the ready light 18.

In accordance with the principles of the present invention an OTC AED is packaged in packaging suitable for purchasers of OTC AEDs as illustrated in FIGS. 3-8. In a constructed embodiment FIG. 3 is the top panel of an OTC AED package, FIG. 4 is the bottom panel of an OTC AED package, FIGS. 5 and 6 are side panels and FIGS. 7 and 8 are end panels of an OTC AED package. Turning first to FIG. 3, the packaging panel 40 there shown contains usual information such as the name of the device (“HeartStart Home Defibrillator”) and its manufacturer (“Philips”). Additionally the panel 40 contains a color picture 42 of the OTC AED contained inside the package. The color picture 42 may be a graphical illustration or other rendering, but in this embodiment the picture is a color photograph of the OTC AED which shows its distinctive coloring. An alternative approach to illustrating the OTC AED on a panel of the packaging is to make the packaged OTC AED visible through the panel as by a clear window or plastic or “blister-pack” covering of the OTC AED which makes the distinctive OTC AED inside the package clearly visible to the layperson purchaser. In the illustrated embodiment the color picture 42 clearly shows the distinctive blue color of the OTC AED inside the package. In this embodiment the OTC AED is packaged with a red carrying case and a color picture of the carrying case 44 appears next to the color picture of the OTC AED. In accordance with a further aspect of the present invention, the carrying case prominently displays a contact number by which the layperson rescuer can reach professional medical assistance. In this example the contact number is the 911 emergency telephone number. In the excitement of a cardiac emergency the rescuer may be focused on using the OTC AED and may not think to promptly call for professional medical assistance, which should be done as soon as possible. Seeing the contact number for professional medical assistance can also help calm the rescuer in what is a very stressful situation. The emergency contact number can be displayed on one side of the case, however in a constructed embodiment the emergency contact number is prominently displayed in a contrasting color (white against red) on multiple sides of the carrying case. It may alternately or additionally be desirable to prominently display an emergency contact number on the OTC AED unit itself.

On the right side of the panel 40 is a list 46 of attributes which are important to the potential purchaser of an OTC AED. In this embodiment the list 46 is directed to the ease of use of the OTC AED, its safety, and its reliability. In accordance with a further aspect of the present invention the OTC AED packaging contains an important notification 47 to the purchaser of the OTC AED, informing him or her of the importance of setting up the OTC AED before it is needed. A purchaser of an OTC AED may take the OTC AED home and leave it in the packaging until it is needed. The notification 47 informs the purchaser of the importance of setting up the OTC AED for use before it is needed, so that the time required to do this does not consume the initial period of a cardiac emergency, when time is of the essence and clear thinking is at a premium. One important function which can be initiated at setup in some embodiments may be the self-test function which, as mentioned above, has a heightened importance in an OTC AED.

In accordance with another aspect of the present invention the panel 40 contains a brief description 48 of the indication of use of the OTC AED. A layperson purchaser of an OTC AED may be unfamiliar with the symptoms which call for an AED and can be confused when confronted with complex medical terminology. The use description 48 clarifies in simple terms what the symptoms of sudden cardiac arrest are (“not responsive [e.g., unconscious] and not breathing normally” in this embodiment) and how the OTC AED responds to this condition (“delivering a shock to the heart”). A potential purchaser of an OTC AED may decide whether to purchase an OTC AED solely based upon a reading of the packaging. It is helpful to provide a use description on the OTC AED packaging to help the potential purchaser make the right decision for his or her circumstances, especially since the correctness of the decision may not become known until the moment of a cardiac emergency.

FIG. 4 illustrates another embodiment of the present invention which is used as the bottom panel 50 of a constructed embodiment. The panel 50 also contains a color picture 52 of the OTC AED inside the packaging. The panel 50 also contains graphically illustrated instructions 54 showing what needs to be done when the OTC AED is put to use. In this embodiment it may be seen that three steps in the use of the OTC AED are shown: pulling the cartridge handle to access the electrode pads, placing the electrode pads on the chest of the patient, and pressing the shock button. A prospective purchase of an OTC AED can thus see what he or she is expected to do in order to use the OTC AED and can factor this information into the purchase decision.

FIGS. 5 and 6 illustrate further embodiments of the present invention. In a constructed embodiment FIGS. 5 and 6 depict the side panels 60 and 70 of packaging for an OTC AED. These side panels 60 and 70 are seen to contain color pictures 62 and 72 of the OTC AED inside the packaging. The side panels also contain color pictures 64 and 74 of the carrying case. By putting color pictures of the OTC AED on the side panels of the packaging, potential purchasers and retail personnel can immediately identify the packages when they are stacked together or stacked with packages of other items. A potential purchaser who pulls the package out from a stack of packages will thus know that he or she is selecting the OTC AED.

FIGS. 7 and 8 illustrate further embodiments of the present invention. In a constructed embodiment the panels of FIGS. 7 and 8 are used as end panels 80 and 90 of packaging for an OTC AED. Panel 80 is seen to give a list 82 of considerations which can help a layperson potential purchaser decide whether to purchase the OTC AED. In this example the first consideration is the information that the OTC AED cannot be used to treat oneself. This is an important consideration for someone living alone, who will thus be alerted that another type of cardiac response may be more suitable, such as an implantable defibrillator or a wearable monitor which constantly monitors the cardiac health of the individual. Other considerations are given in the list 82. The second is the possible need to perform CPR. A third is that it is often necessary to kneel when using the OTC AED, such as is typically done when the patient is unconscious on the floor. Others in the list are that the voice prompts and enclosed instructions are in a particular language (English in this example), and that the unit provides alerts if maintenance is needed. A potential customer who cannot or is uncomfortable with these conditions of use or of the OTC AED inside the package may want to consider a different approach to responding to sudden cardiac arrest.

The panel 90 of FIG. 8 is seen to contain another color picture 92 of the OTC AED and of the carrying case 94. This panel 90 appears as an end panel of the constructed embodiment which thus has color pictures of the OTC AED on five of the six sides of the packaging. A constructed embodiment may have a picture of the OTC AED on every side of the packaging, thereby providing both potential customers and retail establishment employees with an unambiguous indication of the contents of the packaging, no matter how the package is stacked or stored.

When defibrillators are shipped to purchasers the units are shipped without the battery being installed. The units are not shipped with the batteries installed because of the possibility of inadvertent activation and the resultant hazard if the high voltage circuitry begins the charge the defibrillator capacitor to its usual level of hundreds or thousands of volts. It is also possible that a self-test performed during shipment could detect an error condition, causing the defibrillator to issue its audible alerts for maintenance during shipment, a situation to be avoided for obvious reasons such as airline safety. After the defibrillator is received by the purchaser, the first action of the medical professional is to install the battery in the defibrillator, at which point the defibrillator usually performs a self-test known as a “battery insertion test.” This process begins the setup of the defibrillator, which may require periodic intervention by the medical professional before setup is complete. As mentioned above, it is important that the OTC AED be promptly set up when the layperson purchaser takes it home. Furthermore, it is desirable to make setup as simple as possible for the nonmedical layperson. In accordance with a further aspect of the present invention setup of the OTC AED is simplified by providing the OTC AED with its battery already installed, alleviating the layperson of this task. However, to prevent inadvertent charging of the high voltage circuitry and capacitor during shipment, the battery circuit is broken by a nonconductive pull tab 100 during shipment as illustrated in FIG. 9. The distal end 102 of the pull tab 100 is disposed in the battery circuit such as between one battery terminal and its contact on the OTC AED. In a constructed embodiment the battery has four terminals which engage four contacts on the OTC AED, and the distal end 102 is disposed between all four terminals and contacts, completely isolating the battery from the high voltage circuitry of the OTC AED. The pull tab 100 may be made of a sheet of nonconductive material such as paper or cardboard. In a constructed embodiment the pull tab 100 is made of a thin polymeric sheet which is tough enough not to tear when a finger is inserted in the hole 104 in the proximal end of the pull tab and the pull tab is pulled from between the battery terminals and OTC AED contacts. The thin sheet enables the battery to be latched in place in the battery compartment while the pull tab is in place during shipment. The polymeric material also gives the pull tab a resilient property. During shipment in a constructed embodiment the pull tab is folded over the top of the OTC AED when the OTC AED is in the carrying case, and the case is closed. When the case is opened for the first time the resilient pull tab pops up, immediately informing the layperson what is to be done first. The pull tab may be labeled with instructions at its proximal end such as “pull” or “remove first”, or it may be labeled with a graphic such as an arrow pointing up (shown above hole 104), or it may be left unlabeled, with the pop-up characteristic speaking for itself.

It will be appreciated that it is not necessary for the pull tab to pop up when the OTC AED is initially accessed as described above. In another embodiment the pull tab may be prominently positioned so that it is one of the first items seen by the new purchaser when opening the OTC AED package. It is sufficient in most embodiments if the purchaser recognizes the pull tab and understands that it is to be removed.

While the installed battery is a benefit because it alleviates the layperson purchaser of this task, it is also an advantage because the OTC AED packaging does not have to accommodate a separate battery pack and thus can be made smaller. In a constructed embodiment the OTC AED with battery installed and in the carrying case 44 measured 3½″ by 8″ by 9″, a total of 252 in³, and was packed in packaging measuring 6.5″ by 8.375″ by 10.5″, a total of 572 in³. The OTC AED and case thus occupy 44% of the packaging volume.

In accordance with a further aspect of the present invention, the top of the OTC AED is covered with a sheet 110 that obscures from the user certain ones of the controls of the OTC AED, seen in FIG. 1, except for those that are to be used to set up the OTC AED. An embodiment of a sheet 110 is shown in FIG. 10, comprising the top area 112 which covers the top of the OTC AED and contains three instructions: pull the red tab 100 to start the automated setup process; press the orange button indicated by the second arrow when prompted by an audible instruction; and wait until an audible prompt announces that the setup is complete. Two tabs 114 and 116 extend from the top area 112 and fold under the OTC AED, where they are engaged through slits 115 and 117 by a third tab 118, retaining the cover in place around the OTC AED. In a constructed embodiment this sheet comprises removable cardboard packing that covers all but the shock button 26 on the top of the OTC AED 10 as shown in FIG. 11. When the new purchaser opens the carrying case 44 for the first time, the pull tab 100 pops up and the purchaser responds by pulling the tab, connecting the battery terminals to the contacts of the OTC AED. The OTC AED will then immediately commence its battery insertion test, and the audible prompts may announce to the purchaser that testing is underway. At the conclusion of the battery insertion test, during which no user intervention is needed, the purchaser is asked to respond by pressing the shock button 26. At this point no other controls are visible to the purchaser by reason of the cover sheet covering the top of the OTC AED controls except for the shock button. The cover sheet also inhibits the purchaser from pressing any other buttons on the OTC AED while the battery insertion test is in progress. In the illustrated embodiment the cover sheet also obscures the pull handle for the pads cartridge as it is not necessary for the purchaser to pull this handle during setup. When the battery insertion test is complete the purchaser can dispose of the pull tab 100 and the cover sheet 110 and the OTC AED is set up and ready for use in a cardiac emergency.

Purchasers of OTC AEDs may have different individual requirements for storing and training for use of the OTC AED. In accordance with a further aspect of the present invention an OTC AED is sold in multiple accessory configurations. In the most basic configuration the OTC AED is sold in packaging containing only the OTC AED (and carrying case if it has one) and the literature kit for the unit. A typical literature kit includes instructions for setup and use, warranty information, support program reply card as described below, training video, and possibly other information. In the “training” configuration the packaging contains the OTC AED (and case, if appropriate), the literature kit, and a package of training pads. The training pads are used with the OTC AED when the user wants to train himself or others in the use of the OTC AED. In the “emergency healthcare” configuration the packaging contains the items of the training configuration and a first aid kit which the purchaser may also use for home medical emergencies. In the “wall mount” configuration the packaging contains all of the items of the emergency healthcare configuration and a wall mount for mounting the OTC AED on a wall. It will be appreciated that in a given embodiment different mixes of these items will be included and other packaging configurations can be arranged. For example, a configuration of the OTC AED, carrying case, literature kit, training pads and wall mount may be desirable for certain users who already have a first aid kit.

As previously mentioned it is to be anticipated that the consumer purchaser of an OTC AED will be unmindful of any maintenance needs of the OTC AED while it is in its standby state. In accordance with a further aspect of the present invention, a support program for an OTC AED is provided by multiple communications with the purchaser of the OTC AED. To facilitate these communications a mail-in support program card is included in the packaging of the OTC AED, an example of which is shown in FIG. 11. The support program card elicits the purchaser's contact information such as mailing address and telephone number and has a space for the serial number of the OTC AED. The card may also request that the manufacturer be notified if ownership of the OTC AED is transferred to someone else or if the OTC AED is no longer in service. It may also provide a space where the purchaser can state the kind of support desired, such as reminders when the battery or pads need replacing or updates on home defibrillation. In the illustrated embodiment the support program card comes with detachable instructions and offers an incentive if the card is returned as requested.

When the new owner enrolls in the support program and provides the necessary contact information, periodic contacts with the owner may then be conducted. For example, a first such communication can be a letter mailed to the enrolled owner of the OTC AED after approximately the first year of ownership. Such a letter may remind the owner to check the battery and pads of the OTC AED to see if they need replacing, and to set up the OTC AED if that has not already been done. The letter may also offer training opportunities to the purchaser such as reviewing a training video. The letter may also solicit information such as whether the OTC AED was used in an emergency during the past year and whether its ownership or possession have been transferred to another person. A second such communication may occur after two years of ownership, which is approximately the time that pads need replacing. In addition to the foregoing, the letter may remind the owner that the pads of the OTC AED need replacing and may include contact information and an incentive to do so. A similar communication may be sent at the later date when the battery is scheduled for replacement. Such a support program can help keep a potential home rescuer trained to use the OTC AED, can help keep the OTC AED properly maintained, and provides a means to follow ownership of the OTC AED through successive owners so that such services can continue with whomever owns the OTC AED. The contact information also provides a means for the manufacturer to contact the owner if upgrades or improvements or critical information concerning the OTC AED become available in the future.

What is claimed is:

1. A packaged OTC automatic external defibrillator which is sold over-the-counter to a layperson comprising:
an OTC automatic external defibrillator electronic unit;
a battery;
an electrode pad; and
packaging enclosing the OTC automatic external defibrillator electronic unit, the battery, and the electrode pad, the packaging including a plurality of outside panels which include a color illustration of the OTC automatic external defibrillator.

2. The packaged OTC automatic external defibrillator of claim 1, wherein the color illustration comprises a photographic-grade illustration of the OTC automatic external defibrillator.

3. The packaged OTC automatic external defibrillator of claim 2, wherein the photographic-grade illustration of the OTC automatic external defibrillator comprises a photograph of an OTC automatic external defibrillator.

4. The packaged OTC automatic external defibrillator of claim 1, wherein the color illustration comprises a view of the OTC automatic external defibrillator inside the packaging through a transparent portion of the packaging.

5. The packaged OTC automatic external defibrillator of claim 1, wherein the packaging further comprises a box which displays a color illustration of the OTC automatic external defibrillator on at least four sides of the box.

6. The packaged OTC automatic external defibrillator of claim 5, wherein the color illustration comprises a photographic-grade illustration of the OTC automatic external defibrillator.

7. The packaged OTC automatic external defibrillator of claim 6, wherein the photographic-grade illustration of the OTC automatic external defibrillator comprises a photograph of an OTC automatic external defibrillator.

8. The packaged OTC automatic external defibrillator of claim 5, wherein the color illustration comprises a view of the OTC automatic external defibrillator inside the packaging through a transparent portion of the packaging.

9. The packaged OTC automatic external defibrillator of claim 1, wherein the packaging further comprises a box which displays a color illustration of the OTC automatic external defibrillator on at least five sides of the box.

10. The packaged OTC automatic external defibrillator of claim 9, wherein the color illustration comprises a photographic-grade illustration of the OTC automatic external defibrillator.

11. The packaged OTC automatic external defibrillator of claim 10, wherein the photographic-grade illustration of the OTC automatic external defibrillator comprises a photograph of an OTC automatic external defibrillator.

12. The packaged OTC automatic external defibrillator of claim 9, wherein the color illustration comprises a view of the OTC automatic external defibrillator inside the packaging through a transparent portion of the packaging.

13. A packaged OTC automatic external defibrillator which is sold over-the-counter to a layperson comprising:
an OTC automatic external defibrillator; and
packaging enclosing the OTC automatic external defibrillator, the packaging having an exterior indicium identifying the OTC automatic external defibrillator inside the packaging and having an exterior description of one or more indications of a condition for which the use of the OTC automatic external defibrillator may be appropriate, including one or more of patient responsiveness and patient breathing.

14. The packaged OTC automatic external defibrillator of claim 13, wherein the exterior of the packaging further includes a description of the response of the OTC automatic external defibrillator to an indication.

15. The packaged OTC automatic external defibrillator of claim 14, wherein the description of the response comprises a statement that the OTC automatic external defibrillator delivers a shock to the heart.

16. A packaged OTC automatic external defibrillator which is sold over-the-counter to a layperson comprising:
an OTC automatic external defibrillator;
packaging enclosing the OTC automatic external defibrillator; and
a description of one or more considerations which may be important to a layperson when deciding whether to purchase the OTC automatic external defibrillator.

17. The packaged OTC automatic external defibrillator of claim 16, wherein the considerations include one or more of: the OTC automatic external defibrillator may be inappropriate for people with a certain medical condition; and voice prompts are in a given language.
